(19) United States
(12) Patent Application Publication
(10) Pub. No.: US 2025/0161442 A1
(43) Pub. Date: May 22, 2025

(54) TARGETING IL-33 FOR CANCER THERAPY

(71) Applicant: UNIVERSITY HEALTH NETWORK, Toronto (CA)

(72) Inventors: Tracy MCGAHA, Toronto (CA); Sara LAMORTE, Toronto (CA)

(21) Appl. No.: 18/951,569

(22) Filed: Nov. 18, 2024

Related U.S. Application Data
(60) Provisional application No. 63/600,618, filed on Nov. 17, 2023.

(51) Int. Cl. A61K 39/395; A61P 35/00

(52) U.S. Cl. CPC A61K 39/3955; A61P 35/00

(57) ABSTRACT

There is provided herein a method of treating cancer in a subject comprising downregulating IL-33 activity in the subject. Also provided are compositions for treating cancer comprising an inhibitor of IL-33/ST2 interaction and uses thereof.

### RELATED APPLICATIONS

This application claims priority to U.S. Provisional Patent Application No. 63/600,618 filed on Nov. 17, 2024, the entirety of which is incorporated herein by reference.

### FIELD OF THE INVENTION

The invention relates to targeting the IL-33/ST2 axis for cancer therapy.

### BACKGROUND OF THE INVENTION

Cancer therapy (e.g., chemotherapy, radiotherapy, immunotherapy) drives significant cell death, including apoptosis, and the release of tumor antigens. In principle, therefore, chemotherapy should be immunogenic. However, in vivo tumor models and certainly in the clinic it is apparent that chemotherapy drugs do not generate a robust antitumor immune response in the same way that a vaccine or an infection would. We have previously shown that apoptotic cells elicit potent immune suppression and tolerance by mechanisms dependent on indoleamine 2,3 dioxygenase (IDO), general control nonderepressible 2 (GCN2) and Aryl hydrocarbon Receptor (AhR) signals in splenic resident macrophages^{1,2,3,4}. Tumor-draining lymph nodes (TDLN) contain populations of specialized resident macrophages: medullary sinus macrophages, (MSMs); subcapsular sinus macrophages (SSMs), lining the lymphatic sinus and directly exposed to lymph-borne antigens and cellular debris draining through the lymph⁵.

### SUMMARY OF THE INVENTION

In an aspect, there is provided a method of treating cancer in a subject comprising downregulating IL-33 activity in the subject.

In an aspect, there is provided an inhibitor of IL-33/ST2 interaction for use in the treatment of cancer.

In an aspect, there is provided a use of an inhibitor of IL-33/ST2 interaction in the preparation of a medicament for the treatment of cancer.

In an aspect, there is provided a pharmaceutical composition for the treatment of cancer comprising an inhibitor of IL-33/ST2 interaction along with a pharmaceutically acceptable carrier.

In an aspect, there is provided a pharmaceutical composition for the treatment of cancer comprising reagents for genomic editing of the subject's cells to decrease transcription or expression of IL-33.

### BRIEF DESCRIPTION OF FIGURES

These and other features of the preferred embodiments of the invention will become more apparent in the following detailed description in which reference is made to the appended drawings wherein:

FIG. 1. MSMs acquired a tolerogenic phenotype after uptake of ATCs. (A) Representative histograms showing the uptake of TACstdTomato+ by MSMs or SSMs in the draining (red line) versus the contralateral (black line) lymph node 24 h or 48 h after cisplatin treatment. (B) Volcano plot for differential expression based on transcriptome analysis of SSMs and MSMs isolated from the TDLN of cisplatin-treated versus untreated mice. Black dotted line marks FDR <0.05. (C) Heat maps showing modulation of tolerogenic genes in SSMs and MSMs isolated from the TDLN of cisplatin-treated versus untreated mice.

FIG. 2. IL-33 contributes to MSMs tolerogenic phenotype induced by ATCs uptake. (A) A bar plot representing counts per million (CPM) of IL-33 from RNAseq analysis. (B) ELISA for secreted IL-33. (C) Percentage of IL33+ macrophages after in vitro apoptotic cell challenge. (D) Percentage of IL33+ macrophages after in vivo apoptotic cell challenge with or without blocking ATCs uptake (Annexin V).

FIG. 3. IL-33-derived MSMs promotes tumor growth through IL-33/ST2 axis. (A) B16 tumor growth curves in IL33ff and Marco-IL33ko mice after cisplatin treatment. (B) Yumm1.7 tumor growth curves in IL33ff and Marco-IL33ko mice after PLX4720 treatment. (C) B16 tumor growth curves with or without blocking antibody against ST2 (aST2) after cisplatin treatment. (D) Yumm1.7 tumor growth curves with or without blocking antibody against ST2 (aST2) after PLX4720 treatment.

FIG. 4. IL-33-derived MSMs promote increase of suppressive TregST2+. (A) UMAP showing scRNA-seq data of ST2+ cells isolated from IL-33ff and Marco-IL-33ko tumors and their matched TDLN with or without PLX4720 treatment. (B) Percentage of TregST2+ in the TDLN and Tumor. (C) Phenotype of TregST2+ in the TDLN.

FIG. 5. IL-33-derived MSMs inhibits Cd8 T cell activation. (A) Phenotye of Cd8 T cells in the tumor. (B) Yumm1.7 tumor growth curves in Marco-IL33ko mice after PLX4720 treatment in combination with Cd8 depletion antibody (aCD8) every other day.

FIG. 6. Decreased tumor volume with combination therapy. Mice were implanted with BRAF mutant melanoma and given the BRAF inhibitor PLX4720 at the days post tumor insertion indicated. Mice received antibodies against PD1 and the IL-33 receptor (ST2) as 5 i/p injections as indicated. Tumor volumes were monitored (A) and shown at day 28 post tumor introduction (B).

FIG. 7. A schematic showing myeloid cells in the TDLN acquiring an immune-suppressive phenotype as a consequence of exposure to apoptotic tumor cells (ATCs) after cancer therapy, driving tolerance and resulting in increased tumor growth and resistance to therapy.

FIG. 8. B16 tumor growth was measured in MSM-IL33KO or littermate control mice+/−Cytoxan.

FIG. 9. B16 tumor growth was measured in MSM-IL33KO or littermate control mice treated+/−3 doses of irradiation (8Gy).

FIG. 10. MC38 tumor growth was measured in MSM-IL33KO or littermate control mice treated+/−cisplatin.

FIG. 11. UMAP of combined single-cell RNA-seq data from FACS-enriched intratumoral CD45+immune cells from MSM-IL33KO and littermate control mice+/−7× treatments with PLX4720.

FIG. 12. Representative bioluminescence image (left) and quantification the total flux of the individual mice±SD (right) of Yumm tumor-bearing+/−PLX4720 in combination with aST2 IgG with primary tumor excised and rechallenged with 10⁶ YUMM1.7^{LUC} cells i.v. The graph on the right shows data pooled from two different experiments.

FIG. 13. Yumm1.7 tumor bearing mice were treated with PLX4720+/−αPD1 IgG and/or αST2 IgG. Graph shows mean tumor volume at day 38 post-implantation. For tumor growth curve in (I) and bar graph in (J) significance was determined by two-way ANOVA. For other bar graphs significance was determined by unpaired students T test with ***=pval<0.001 and ****=pval<0.0001. All experiments were 1592 repeated at least twice with similar results.

FIG. 14. CD8+ T cell clusters in the TDLN and the impact of combining ST2 inhibition with checkpoint inhibitor therapy in melanoma. YUMM tumor growth was measured in B6 mice+/−PLX4720 in combination with αPD1 IgG or αST2 IgG blocking antibodies. The crosses indicates groups that were sacrificed due to tumor burden. For tumor growth each data point represents average tumor volumes+/−SD and significance was determined by two-way ANOVA. All experiments were repeated 3 times with similar results.

FIG. 15. B16 tumor growth was measured in Foxp3^{GFPDTR} mice depleted of endogenous Treg and receiving adoptive transfer i.v. of 10⁶ splenic Treg cells isolated from either ST2^{WT} or ST2^{KO} mice and then treated with Cisplatin.

### DETAILED DESCRIPTION

In the following description, numerous specific details are set forth to provide a thorough understanding of the invention. However, it is understood that the invention may be practiced without these specific details.

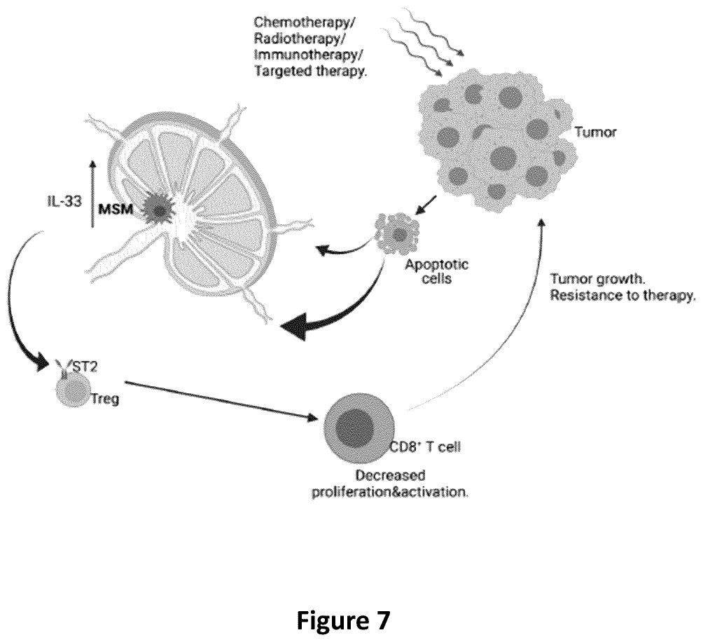

Referring to FIG. 7, we show that after cancer therapy, myeloid cells in the TDLN acquire an immune-suppressive phenotype as a consequence of exposure to apoptotic tumor cells (ATCs), driving tolerance and resulting in increased tumor growth and resistance to therapy.

In an aspect, there is provided a method of treating cancer in a subject comprising downregulating IL-33 activity in the subject.

As used herein, the term “downregulating” refers to at least partial inhibition, neutralization, or knockdown of the expression of a gene or an activity of the protein that it encodes. For example, the downregulating may comprise administering to the subject an antibody or inhibitor against IL-33 or ST2 or knocking down either gene.

In some embodiments, the method of treating cancer in a subject comprises downregulating IL-33 activity in the subject in combination with a cancer therapy.

The cancer therapy may be chemotherapy, radiotherapy, immunotherapy, PD-1 blockade, targeted therapy, or any combination thereof.

In some embodiments, downregulating IL-33 activity in the subject comprises administering an inhibitor of IL-33/ST2 interaction to the subject.

In some embodiments, the inhibitor is an antibody against ST2.

In other embodiments, the inhibitor is an antibody against IL-33.

The terms “antibody” and “immunoglobulin”, as used herein, refer broadly to any immunological binding agent or molecule that comprises an antigen binding domain, including polyclonal and monoclonal antibodies. Depending on the type of constant domain in the heavy chains, whole antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

Generally, where whole antibodies rather than antigen binding regions are used in the invention, IgG and/or IgM are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

As will be understood by those in the art, the immunological binding reagents encompassed by the term “antibody” extend to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)2, single domain antibodies (DABs), T and Abs dimer, Fv, scFv (single chain Fv), dsFv, ds-scFv, Fd, linear antibodies, minibodies, diabodies, bispecific antibody fragments and the like.

The techniques for preparing and using various antibody-based constructs and fragments are well known in the art.

Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art.

The antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

Preferably, the antibody or antibody fragment comprises an antibody light chain variable region (VL) that comprises three complementarity determining regions or domains and an antibody heavy chain variable region (VH) that comprises three complementarity determining regions or domains. Said VL and VH generally form the antigen binding site. The “complementarity determining regions” (CDRs) are the variable loops of β-strands that are responsible for binding to the antigen. Structures of CDRs have been clustered and classified by Chothia et al. (J Mol Biol 273 (4): 927-948) and North et al., (J Mol Biol 406 (2): 228-256). In the framework of the immune network theory, CDRs are also called idiotypes. The antibodies are preferably human antibodies. Methods for humanizing antibodies are also know in the art.

In still other embodiments, the inhibitor is a small molecule against ST2 or IL-33.

Some such small molecule inhibitors are known in the art, as described in Mai et al.,⁷ Ramadan et al.⁸ and Le et al.⁹

In some embodiments, downregulating IL-33 comprises genome editing of the subject's cells to decrease transcription or expression of IL-33.

“Genome editing”, or genome engineering, or gene editing, as used herein refers to genetic engineering in which DNA is inserted, deleted, modified or replaced in the genome of a living organism. Genome editing targets the insertions to site-specific locations. Methods for genome editing in the art include zinc-finger nucleases (ZFNs), transcription activator-like effector nucleases (TALENs), meganucleases and CRISPR/Cas9 systems.

In some embodiments, the cancer is Acute lymphoblastic leukemia (ALL), Acute myeloid leukemia (AML), Adrenal gland, Anal, Bile duct, Bladder, Bone, Brain and spinal cord, Breast, Cervical, Chondrosarcoma, Chordoma, Chronic lymphocytic leukemia (CLL), Chronic myeloid leukemia (CML), Colorectal, Esophageal, Eye, Fallopian tube, Gallbladder, Gestational trophoblastic disease, GISTs, Hodgkin lymphoma, Hypopharyngeal, Kaposi sarcoma, Kidney, Laryngeal, Leukemia, Liver, Lung, Merkel cell carcinoma, Mesothelioma, Metastatic, Multiple myeloma, Myelodysplastic syndromes, Myeloproliferative neoplasms, Nasal and paranasal sinus, Nasopharyngeal, Neuroblastoma, Neuroendocrine tumours, Non-Hodgkin lymphoma, Oral, Oropharyngeal, Osteosarcoma, Ovarian, Pancreatic, Parathyroid, Penile, Pituitary gland tumours, Prostate, Renal pelvis and ureter, Retinoblastoma, Rhabdomyosarcoma, Salivary gland, Skin—melanoma, Skin—non-melanoma, Small intestine, Soft tissue sarcoma, Stomach, Testicular, Thymus, Thyroid, Uterine, Vaginal, Vulvar, or Wilms tumour, preferably melanoma.

In an aspect, there is provided an inhibitor of IL-33/ST2 interaction for use in the treatment of cancer.

In an aspect, there is provided a use of an inhibitor of IL-33/ST2 interaction in the preparation of a medicament for the treatment of cancer

In an aspect, there is provided a pharmaceutical composition for the treatment of cancer comprising an inhibitor of IL-33/ST2 interaction along with a pharmaceutically acceptable carrier.

In some embodiments, the pharmaceutical composition further comprises one or more agents for chemotherapy, radiotherapy, immunotherapy, PD-1 blockade, and targeted therapy.

As used herein, “pharmaceutically acceptable carrier” means any and all solvents, dispersion media, coatings, antibacterial and antifungal agents, isotonic and absorption delaying agents, and the like that are physiologically compatible.

Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

In an aspect, there is provided a pharmaceutical composition for the treatment of cancer comprising reagents for genomic editing of the subject's cells to decrease transcription or expression of IL-33.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The advantages of the present invention are further illustrated by the following examples. The examples and their particular details set forth herein are presented for illustration only and should not be construed as a limitation on the claims of the present invention.

### EXAMPLES

### Methods and Materials

### Mouse Model

A targeting construct was designed to express enhanced green fluorescent protein (EGFP) and bacteriophage P1 Cre recombinase (Cre) containing a nuclear localization signal (NLS) at the N-terminus under the control of the endogenous mouse macrophage receptor with collagenous structure (mMarco) promoter. The construct design preserves the complete endogenous mouse Marco gene including the endogenous promoter, intron-exon structure and 3′UTR and uses an internal ribosome entry site (IRES) to express an EGFP and NLS-Cre fusion protein from the region just 3′ of the endogenous mouse Marco stop codon. To generate the genomic fragments for the 5′ and 3′ homology arms, we designed primers based on the mouse Marco genomic sequence (GenBank Accession No. NC_000067) to use in PCR from mouse C57BI/6 genomic DNA (Jackson Laboratory, Bar Harbor, Maine). In brief, PCR primers 5′-CTG TCG ACT CTA CCA GCC ATA TCC TCC TGT ACA TTC-3′ (mMarco_Sall sense) and 5′-CGA ATT CCC AAG TCA GGA GCA TTC CAC ACC CGC ATC-3′ (mMarco_EcoRI antisense) were used to amplify a 3539 bp 5′ homology-arm fragment (corresponding to a genomic fragment encompassing mMarco intron 15, exon 16, intron 16 and exon 17 to the endogenous “TGA” stop codon) from 200 ng of C57BI/6J genomic DNA using the Phusion Hot Start Flex DNA Polymerase (New England Biolabs). Using the same PCR conditions, a 2744 bp 3′ homology-arm fragment (encompassing the genomic region 3′ of the mMarco 3′UTR) was amplified from C57BI/6J Genomic DNA using the PCR primers 5′-AGG TTT AAA CAG ATG AGT CTG AAG TGT GTC AAA GTT ACT G-3′ (mMarco Pmel sense) and 5′-AAG CGG CCG CAT TAA GAC ACT GTA GTC TCT GCT CTC AG-3′ (mMarco Notl antisense). pMSCV-Puro IRES EGFP-NLS-Cre (Addgene plasmid #50935) was used as a template to generate the IRES, EGFP, and NLS-Cre fragments using Phusion Hot Start Flex PCR as described below. The 590 bp internal ribosome entry site (IRES) from the encephalomyocarditis virus (EMCV) was amplified with the following PCR primers: 5′AGC TAG CCC CCC CCC CTA ACG TTA CTG GCC GAA 3′ (Nhel IRES sense) and 5′ TAG AGC TCA TTA TCA TCG TGT TTT TCA AAG G 3′(Sacl IRES antisense). The EMCV IRES was chosen since it is the most widely used IRES sequence and is active in a variety mammalian cell lines with the average expression of ECMV IRES-dependent coding regions usually ranging from 20 to 50% that of the first gene in the polycistronic mRNA (Mizuguchi et al., 2000). Enhanced green fluorescent protein (codon optimized for mammalian expression) was amplified from pMSCV-Puro IRES EGFP-NLS-Cre using the primers: 5′ TGA GCT CTA CGC CAC CAT GGT GAG CAA GGG 3′(EGFP Kozak Sacl sense) and 5′ CCG CGG CTG AGC CTC CAC CAG ATC CGC CTC CGC TTG CGG CCT TGT ACA GCT CGT CCA TGC CGA 3′(EGFP GGGSx2 SacII antisense). This 772 bp fragment was engineered with a Kozak consensus sequence upstream of the start methionine and a flexible linker (GGGSx2) was inserted in-frame at the c-terminus to allow independent folding of the EGFP and NLS-Cre within the fusion protein.

To investigate the impact of immune tolerance induced by apoptotic tumor cells within secondary lymphoid organs, we utilized a melanoma model. In this model, we injected B16F10, B16F10^{IL33ko} or Yumm1.7^{IL33ko} cell lines (respectively, 300×10⁴ and 200×10⁴ cells/mouse) subcutaneously. When the tumor reached around 20 mm³, mice were treated with either the chemotherapeutic agent cisplatin (10 mg/kg) or the targeted agent PLX4720 (Braf inhibitor, 20 mg/kg). Subsequently, we assessed the influence of cancer therapy in the TDLN and as control, in the CNLN, which is located in the same anatomical position but does not receive lymph drainage from the tumor. In some experiments, mice were treated with an antibody depleting CD8 (clone 2.43, BioXCell, 150 μg/mouse)

For in vivo ATC challenge, mice were injected in the foot pad with 20 ul of 10×10\hat{ }6 ATCs in a total volume of 20 μL of PBS.

To investigate the impact of immune tolerance induced by apoptotic tumor cells within secondary lymphoid organs, we utilized a melanoma model. In this model, we injected Yumm1.7^{IL33ko} cell lines (200×10⁴ cells/mouse) subcutaneously. Once the tumor reached around 20 mm³, mice were treated with either the targeted agent PLX4720 (Braf inhibitor, 20 mg/kg) alone or in combination with anti-ST2 (clone DIH4, Biolegend) and anti-PD1 (clone RMP1-14, BioxCell) at a concentration of 150 μg/mouse through intraperitoneal injection every other day. Tumor volumes were monitored.

### Generation of IL33 Crispr Cell Lines

B16F10 or Yumm1.7 cells were cultured respectively with DMEM high glucose or DMEM/F12 medium supplemented with 10% FBS and 1% v/v penicillin-streptomycin (Invitrogen, Carlsbad, CA, USA). When cells (in 1 well of a 12-well plate) reach about 60-70% confluence, 0.5 μg from 3 different IL33-gRNA (pSpCas9BB-2A-GFP, GenScript) were co-transfected with lipfectamine3000 reagent (Invitrogen, Carlsbad, CA, USA). After 24 hours, the cells were trypsinized and sorted into 96-well plate based on positive-GFP expression. Then the culture medium was refreshed and supplemented with 6 μg/mL blasticidin antibiotics for removal of transient transfection cell clones the next day. After one week of culture and selection, the survival clones were propagated for functional analysis.

### Tumor Sample Preparation and Flow Cytometry

Lymph nodes and tumors were digested and homogenized in presence of 100 U/ml collagenase IV and 50 U/ml DNase I in complete RPMI medium at 37° C. For analysis of intracellular cytokine production, cells were incubated with GolgiStop (eBioscience) for 4 hours and then washed and fixed/permeabilized with permeabilization/fixation buffer (eBioscience). For flow cytometric analysis, at least 10⁵ events were collected on the LSRFortessa Flow Cytometer (BD Biosciences). Data were analyzed by FlowJo (Tree Star Inc.).

### Bulk RNA-Seq Analysis

TDLN from three mice per condition were digested, pooled, and stained with anti-CD11b, anti-CD11c, anti-CD169, anti-SIGNR1, anti-CD3, anti-CD19 antibodies and 4′,6-diamidino-2-phenylindole. Live CD11B⁺CD11c^{low}CD169⁺SIGNR1⁺CD3⁻CD19⁻ (MSMs) or CD11B⁺CD11c^{low}CD169⁻SIGNR1⁺CD3⁻CD19⁻ (SSMs) were FACS-sorted into RL-buffer (Norgen). RNA was purified (Norgen) and quantified by qubit (Life Technologies) and an Agilent Bioananlyzer assessed the RNA quality. All samples had RIN above 8. Libraries were prepared using TruSeq Stranded mRNA kit (Illumina). Two hundred ng of total RNA were purified for polyA tail containing mRNA molecules using poly-T oligo attached magnetic beads, following purification RNA was fragmented. The cleaved RNA fragments were copied into first strand cDNA using reverse transcriptase and random primers. This was followed by second strand cDNA synthesis using RNAse H and DNA Polymerase I. A single “A” base was added, and adapter ligated followed by purification and enrichment with PCR to create cDNA libraries. Final cDNA libraries were size validated using an Agilent Bioanalyzer and concentration validated by qPCR. All libraries were normalized to nM and pooled together. Pooled libraries were further diluted to 2 nM and denatured with 0.2N NaOH.

1.7 μM of pool libraries were loaded onto a Nextseq cartridge for cluster generation and sequenced Pair-end 75 cycles V2 using Illumina Nextseq500 to achieve a minimum of ˜35 million reads per sample.

A total of 1,325,194,544 sequencing reads were obtained in the four conditions, with each condition sequenced as a biological triplicate. After de-multiplexing and initial quality control, all sequencing reads were aligned against the mouse genome reference sequence GRCm38 with STAR v2.5.1a 6. We used the inherited 5′ _trimming method of STAR as well as the inherited read counting. The entire command was as follows: STAR-outFilterMismatchNoverLmax 0.05-outFilterType BySJout-outSAMstrandField intronMotif-outSAMattributes NH HI AS nM NM MD XS-outSAMmapqUnique 60-quantMode GeneCounts-outFilterintronMotifs RemoveNoncanonical-outFilterMultimapNmax 1-clip5pNbases 13-sjdbGTFfile $GRCm38.ensembl85.gtf. Read counts were measured for gene annotations downloaded from Ensembl Genes V85 7. This resulted in a total of 990,489,880 (74.74%) of overall reads mapping as pairs to annotated genes. Next, inter-sample normalization for read counts was applied with edgeR 8 resulting in counts per million (CPM). After filtering for lowly expressed genes (logCPM>0 across all three replicates of at least one condition), normalized read counts were used for a differential gene expression analysis again using edgeR. We corrected for multiple testing, reporting the false discovery rate (FDR).

### Enzyme-Linked Immunosorbent Assay

TDLN were mechanically disaggregated in 1 ml of PBS, cells were centrifuged at 400 g per 5 minutes and supernatants were collected. Enzyme-linked immunosorbent assays (ELISAs) were performed using kits from R&D (catalog no. DY3626) according to the manufacturer's instructions. The plates were read using a BioTek microplate reader at a wavelength of 450 nm.

### Culture of Macrophages, Generation of Tumor Apoptotic Cells and Co-Culture

Resident macrophages were obtained from 6-8-week-old female mice by peritoneal lavage with 3 ml ice cold PBS and were cultured in RPMI 1640+10% fetal bovine serum (FBS), 100 U/ml penicillin and streptomycin (ThermoFisher).

B16F10^{IL33KO} cell lines were treated with 200 ug/ml of cisplatin for 8 h at 37 C in RPMI+2% FBS to generate ATCs. Flow cytometry analysis revealed >90% cells were apoptotic and less than 1% were necrotic as determined by annexin V and propidium iodine staining. In some experiments ATCs were treated with annexin V for 30 minutes prior to injection to mask phophatidylserine (PS).

Peritoneal macrophages were incubated with ATCs at a ratio of 1:5 for 4 hours, cells were then washed twice with warmed RMPI+10% FBS to remove any ATCs unbound to the macrophages. Fresh media was added, and co-culture was left untouched for 16 hrs before harvesting the cells and media for further analysis.

scRNA-Seq Analysis

Tumors and matching TDLN from three IL33^{fl/fl} or 3 Marco-cre IL33ko mice were digested, pooled, and stained with anti-CD45.2, anti-ST2 antibodies and 4′,6-diamidino-2-phenylindole. Live CD45+ST2+ cells were FACS-sorted into buffer (PBS+2% FBS), washed twice with PBS+0.04% BSA, and then mixed with 10× Genomics Chromium single-cell RNA master mix, followed by loading onto a 10× Chromium chip according to the manufacturer's protocol to obtain single-cell cDNA. Libraries were subsequently prepared and sequenced using the HiSeq 2500 sequencer (Illumina).

### Statistical Analysis

Means, SDs, and unpaired Student's t test results were used to analyze the data. Tumor growth was analyzed using two-way analysis of variance (ANOVA). When comparing two groups, P≤0.05 was considered to be significant.

### Results and Discussion

MSMs Acquire a Tolerogenic Phenotype after Uptake of ATCs.

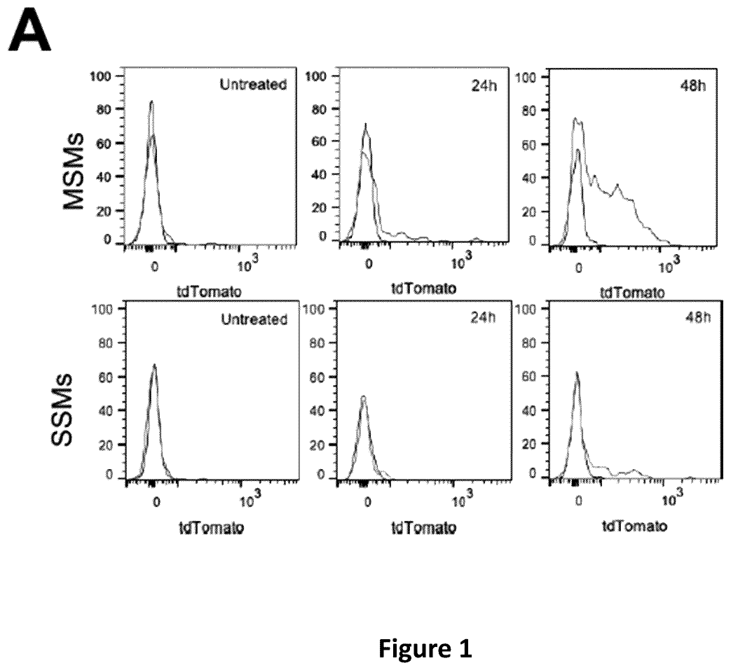

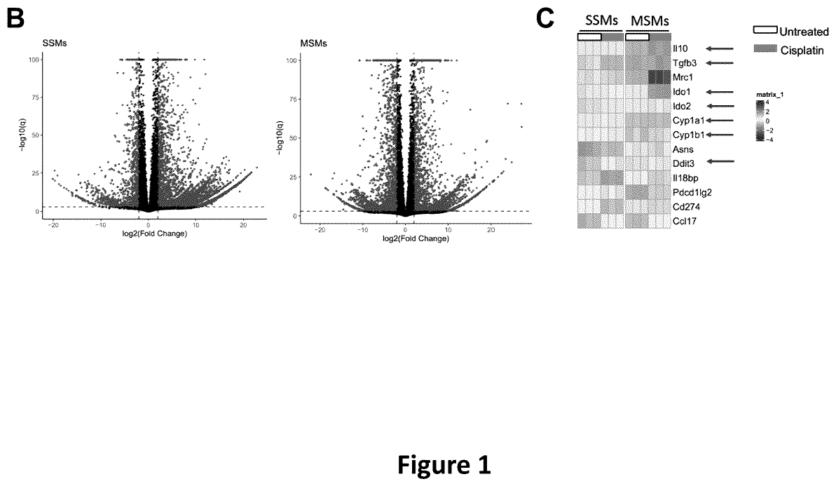

We decided to investigate the impact of immune tolerance induced by apoptotic tumor cells within secondary lymphoid organs. First, we tracked the uptake of ATCs in the TDLN following chemotherapy. In the TDLN of B16F10^{tdTomato+} bearing mice 48 h after chemotherapy treatment, we detected tdTomato signal in both MSMs and SSMs compared to the contralateral lymph node (CNLN) (FIG. 1A). This data indicated that both populations of macrophages were capable of up taking ATCs. Our previous studies, have demonstrated that efferocytosis induced a transition towards a tolerogenic phenotype, characterized by reduced expression of genes involved in the inflammatory response and increased expression of anti-inflammatory genes (1-4). Therefore, using RNA-seq analysis, we assessed the phenotype of both MSMs and SSMs following chemotherapy. To account for any potential effects of the treatment, we also included cells isolated from lymph nodes of untreated and chemotherapy-treated tumor-naïve mice. Overall, we found that 48 h after cisplatin, both MSMs and SSMs exhibited substantial modulation of their gene expression, affecting more that 5000 genes (FIG. 1B). Interestingly, an in dept comparison of MSM versus SSM transcriptome, revealed a decreased expression of pro-inflammatory cytokines such as IL-6 and IL-1b, and increased expression of anti-inflammatory genes like IL-10, Tgfb3 as well as upregulation of key enzymes involved in the activation of tolerogenic pathways, including indoleamine 2,3 dioxygenase (IDO1 and 2); DNA damage inducible transcript 3 (Ddit3); cytochrome P450 family 1 subfamily A member 1 (Cyp1a1) and cytochrome P450 family 1 subfamily B member 1 (Cyp1b1) both belonging to the aryl hydrocarbon receptor pathway (FIG. 1C). These collective data suggested that MSMs, but not SSMs, exhibited a shift towards a tolerogenic phenotype following cisplatin treatment.

### IL-33 Contributes to MSMs Tolerogenic Phenotype Induced by ATCs Uptake.

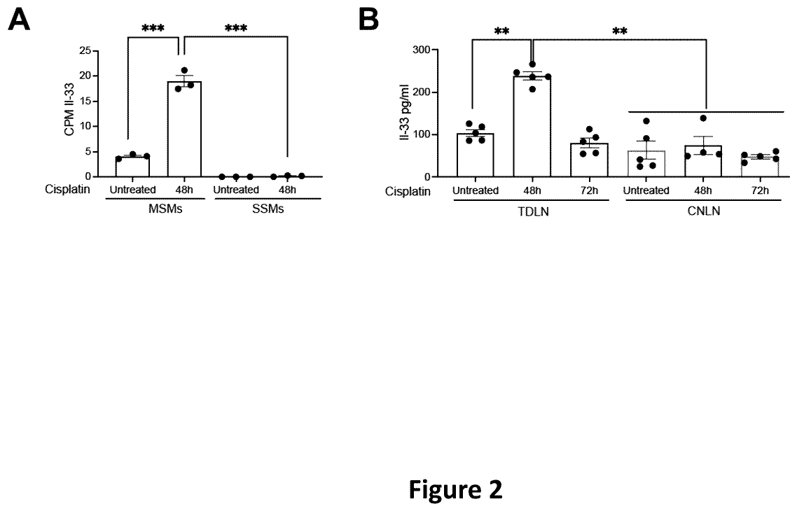

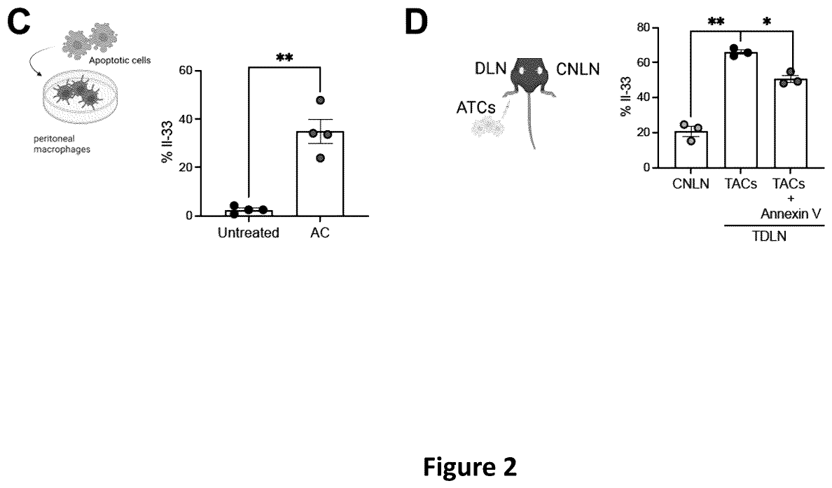

We were interested in investigating new genes contributing to MSM tolerogenic phenotype following ATC uptake. Interestingly, our RNA-seq data showed that 48 h after chemotherapy, MSMs significantly upregulated IL-33 production and that this upregulation occurred exclusively in MSMs (FIG. 2A). This observation prompted us to explore the role of IL-33 production by MSMs. To eliminate the potential influence of tumor-derived IL-33 in our melanoma model, we utilized IL-33-CRISPR knockout melanoma cell lines in all our subsequent in vivo experiments. Initially, we validated the elevated IL-33 expression following cisplatin by ELISA (FIG. 2B) following cisplatin treatment. Moreover, using an in vitro assay, we showed an increase of peritoneal macrophages expressing IL-33 after stimulation with in vitro generated ATCs (FIG. 2C). To investigate the potential correlation between efferocytosis and IL-33 induction in MSMs, we challenged mice with in vitro-generated ATCs, either with or without pre-treatment with Annexin V to inhibit their uptake and clearance by M$. The results showed that pre-treatment with Annexin V impaired the ability of ATCs to induce IL-33 production (FIG. 2D). These data suggested that IL-33 is induced following efferocytosis and that might be involved in the acquisition of a tolerogenic phenotype.

### IL-33-Derived MSMs Promotes Tumor Growth Through IL-33/ST2 Axis.

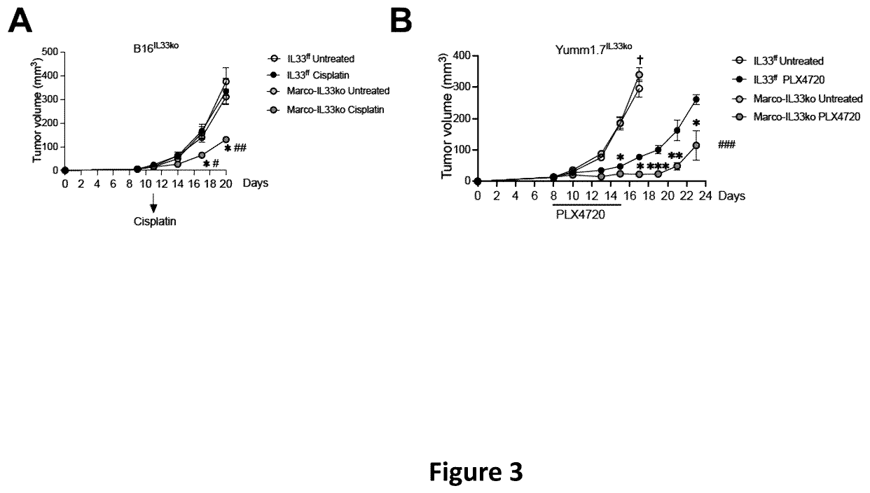

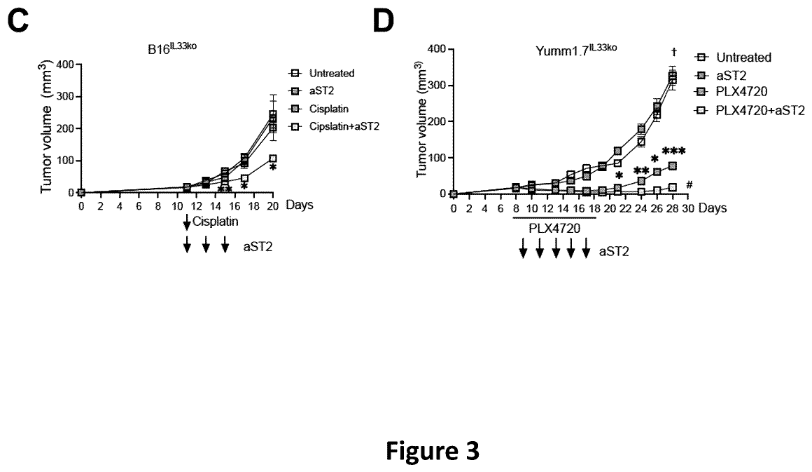

To investigate the role of IL-33 production by MSMs in promoting tumor growth we created a novel Cre-lox system where Cre recombinase is expressed under the promoter of Marco (referred to as Marco-IL33ko) (see Methods and Materials). Using Marco-IL33ko mice we investigate the impact of IL-33 after cancer therapy challenge on tumor growth. Under untreated conditions, Marco-IL33ko mice showed no significant difference in tumor growth compared to IL33^{ff} mice (FIG. 3A, B). However, a substantial impairment in melanoma growth was observed after either chemotherapy (cisplatin) or targeted therapy (PLX4720) (FIG. 3A, B). Suggesting that IL-33 produce by MSMs in the TDLN acted as a limiting factor for the efficacy of cancer therapy. To further validate these data, we employed a pharmacological approach to block the activation of IL-33 target cells using a blocking antibody against ST2 (aST2). ST2 blockade resulted in an enhanced therapeutic effect of both cisplatin and PLX4720 leading to a drastic reduction in tumor growth (FIG. 3C, D). All the above data suggests that MSMs contribute to tumor growth through the IL-33/ST2 axis triggered following cancer therapy.

### IL-33-Derived MSMs Promote Increase of Suppressive TregST2+.

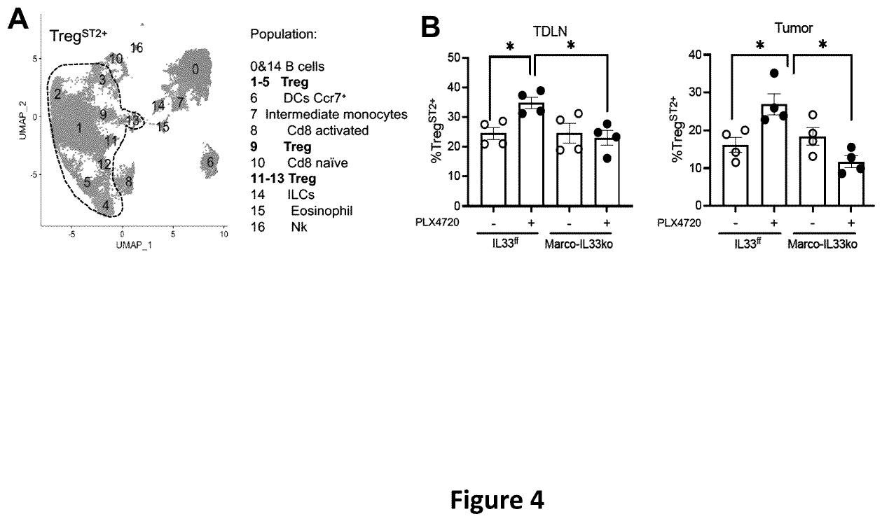

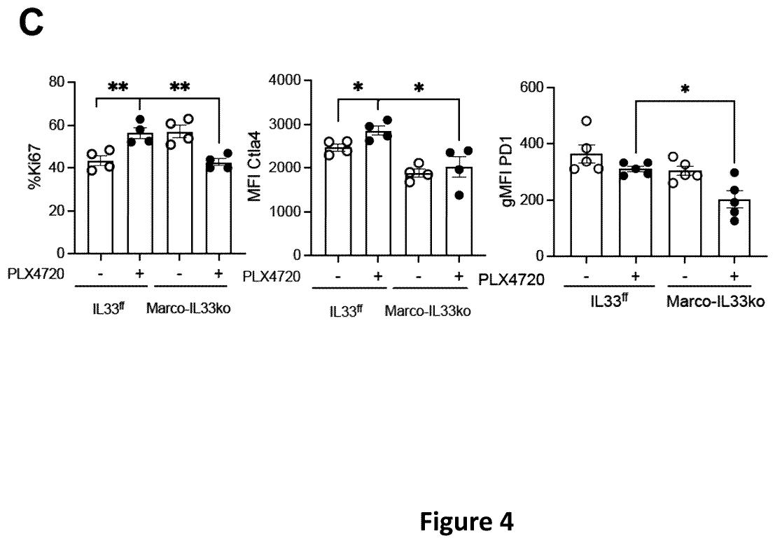

We investigated which cells represented the target of MSM-derived IL-33 in our model. To do so, we performed single-cell RNA-seq (scRNA-seq) on CD45⁺ST2⁺ cells sorted from the melanoma tumor and matched TDLN in IL33^{ff} and Marco-IL33ko mice after a single PLX4720 treatment. Phenograph analysis of the combined data from our experimental groups and tissues showed that the Cd45⁺ST2⁺ population consisted of 16 cell clusters in the TDLN (FIG. 4A). The majority of cells was represented by Treg (Cd4⁺Foxp3⁺Cd25⁺) (FIG. 4A). Flow cytometer analysis confirmed an increase of Tregs^{ST2+} in both melanoma tumors and their matched TDLN after PLX4720 treatment, an increase absent in Marco-IL33ko mice (FIG. 4B). Moreover, in TDLN Tregs^{ST2+} from Marco-IL33ko mice showed a less immunosuppressive phenotype characterized by a decrease in co-inhibitory molecule expression such as Ctla4 and PD1 as well as a decreased proliferation (Ki67) compared to IL33^{ff} (FIG. 4B). Taken together, these results suggest that suggesting that interfering with the IL33/ST2 signaling pathway, in combination with cancer therapy, modulated Treg^{ST2+} function.

### IL-33-Derived MSMs Inhibits Cd8 T Cell Activation.

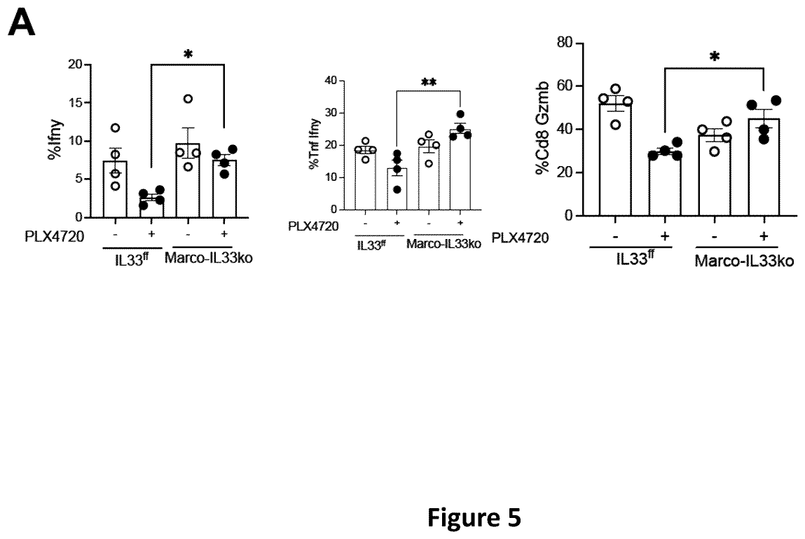

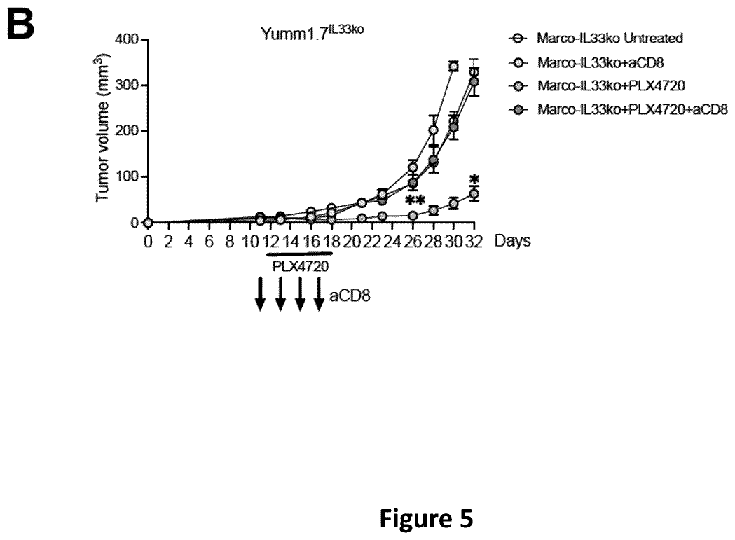

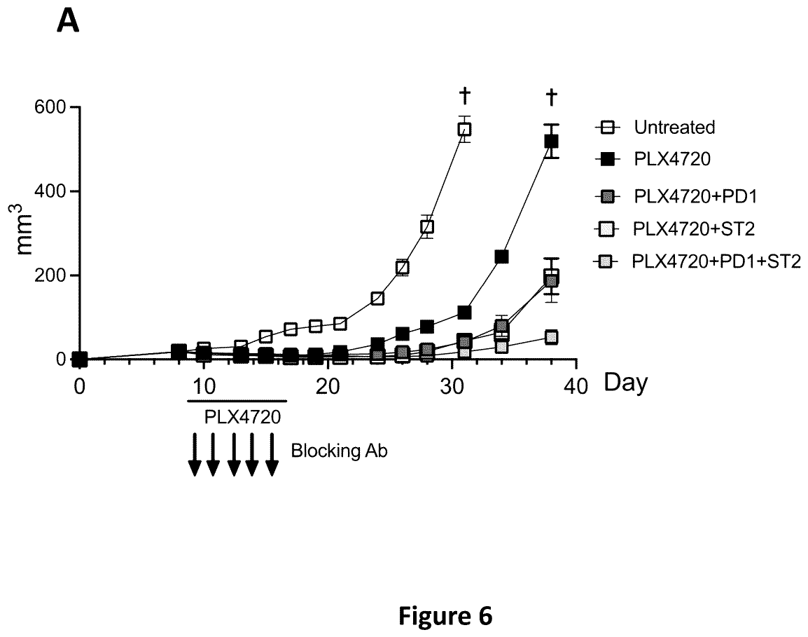

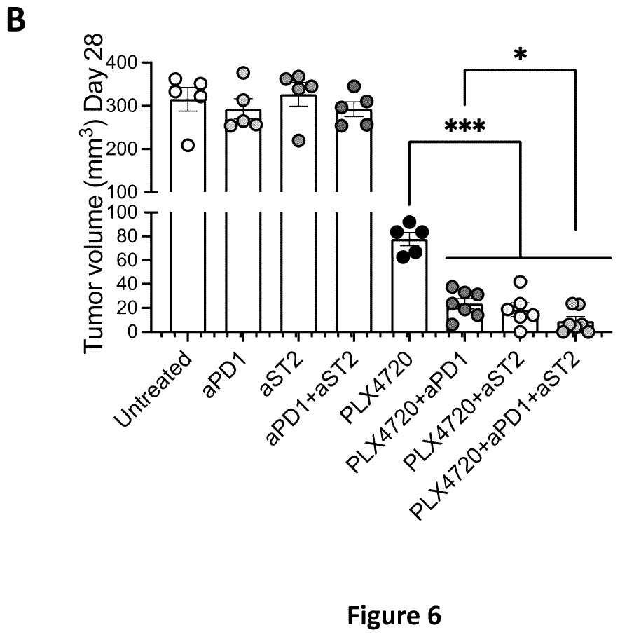

The above data suggested that MSM-derived IL-33 promoted Treg^{ST2+} function, which might be consequently translated into inhibition of CD8 T cells. To substantiate this hypothesis, we analyzed the phenotype of Cd8 T cells in Marco-IL33ko and IL33^{ff} mice, following targeted therapy. Our results showed an increase percentage of Ifnγ⁺, Ifnγ⁺Tnf⁺ and Gzmb⁺ in Cd8 T cells of Marco-IL33ko versus IL33^{ff} mice following PLX4720 treatment (FIG. 5A). Moreover, depletion of Cd8 T cells in combination with PLX4720 treatment rescued the reduced tumor growth effect of Marco-IL33ko (FIG. 5B). Collectively, these findings suggested that MSM-derived IL-33 ultimately influenced Cd8 T cells activation.

### IL-33/ST2 Axis Limit the Efficacy of Immunotherapy.

Our preliminary results suggested that IL-33/ST2 axis promotes the growth of the tumor after cancer therapy. Cancer therapies are often combined with immunotherapy to improve overall survival (1). Therefore, we investigated the role of IL-33/ST2 in tumor progression using aST2 in combination with the anti-programmed cell death protein 1 (PD1) blocking antibody (aPD1). Our results showed that the combination of PLX4720, aST2, and aPD1 improved the overall survival compared to only PLX4720, aPD1 or aST2 alone.

### MSM IL-33 Production is a Key Factor Limiting Tumor Responses to Multiple Therapeutic Modalities in Varied Tumor Types

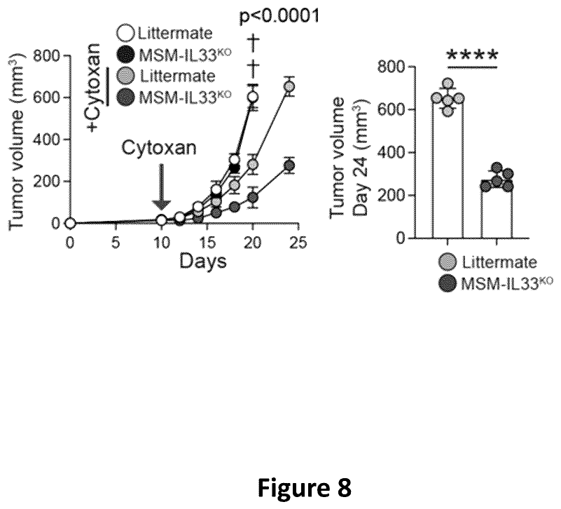

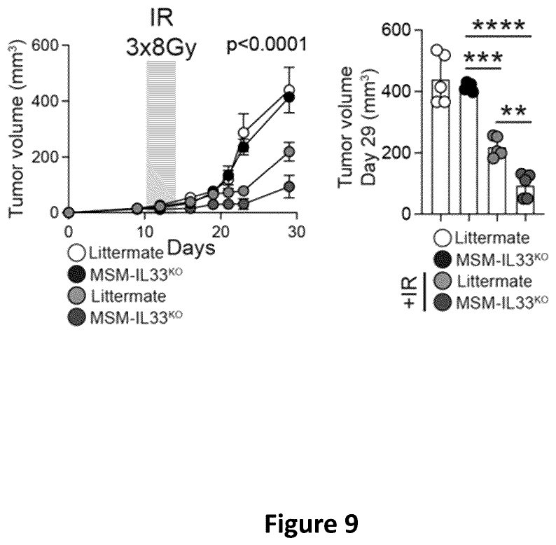

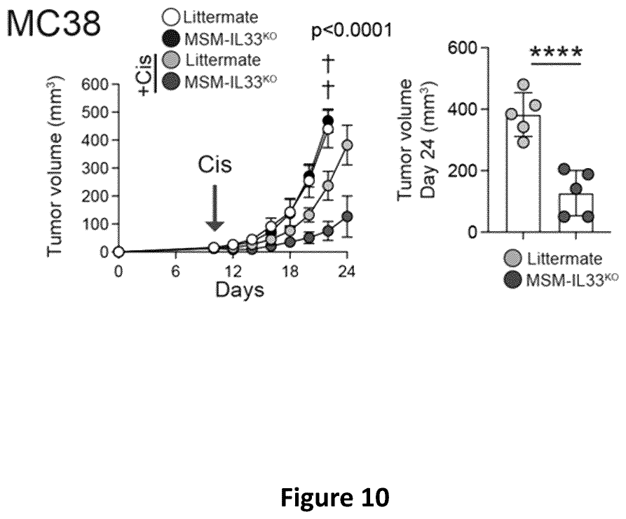

To more broadly test the impact of MSM produced IL-33 in tumor responses to therapy we also examined B16 tumor-bearing MSM-IL33^{KO} mouse responses to Cytoxan (i.e. cyclophosphamide) or radiation therapy. A single dose of Cytoxan (100 mg/kg) 10 d after tumor implantation improved survival in littermate controls compared to untreated groups (that were sacrificed at 20 d due to tumor burden, FIG. 8). However, at 24 d tumor burden in MSM-IL33^{KO} mice was reduced by greater than 50% compared to littermate Cytoxan-treated mice (mean volume of 653 mm³+/−46 versus 276 mm³+/−39 in littermate control and MSM-IL33^{KO} groups respectively) suggesting enhanced tumor control in the absence of MSM IL-33. We saw similar responses to radiation therapy. Three doses of 8 grays (1 treatment per day over 3 days) beginning 10 d post tumor transplantation was sufficient to reduce tumor burden 2-fold compared to untreated controls (FIG. 9). This effect was enhanced in MSM-IL33^{KO} mice where tumor burden was reduced by greater than 2-fold compared to littermate controls receiving radiation therapy (FIG. 9). Finally, we tested if MSM IL-33 contributed to therapy resistance in other tumor models. Implantation with 2×10⁵ MC38 colon carcinoma cells resulted in rapid tumor growth in both MSM-IL33^{KO} and littermate control mice (FIG. 10). Treatment of littermate control tumor bearing mice with cisplatin reduced tumor burden compared to untreated groups; however, the reduction was significantly more pronounced in cisplatin-treated MSM-IL33^{KO} mice which exhibited a 3-fold reduction in tumor burden compared to cisplatin treated controls (127 mm³+/−74 versus 382 mm³+/−71 respectively). Thus the data show that MSM IL-33 production is a key factor limiting tumor responses to multiple therapeutic modalities in varied tumor types.

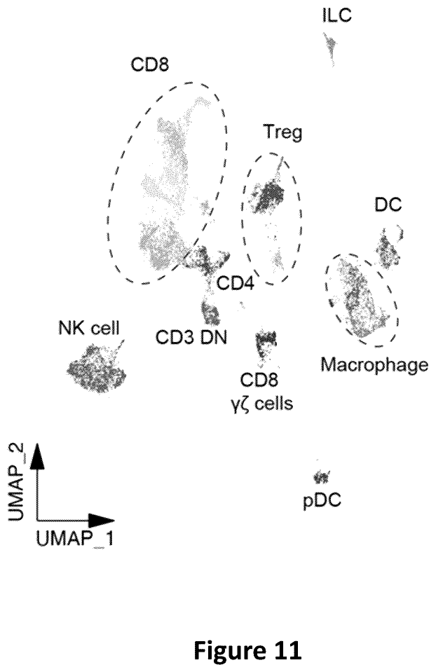

Our data shows that loss of MSM IL-33 production causes significant changes after PLX4720 therapy in the tumor, prolonging tumor regression after treatment. To understand the underlying transcriptional and cellular changes driving this effect, we enriched the CD45⁺ immune cells from YUMM tumors after PLX4720 treatment by FACS and analyzed approximately 60,000 cells by scRNA sequencing analysis. The largest proportion of intratumoral immune cells were CD8⁺ T cells although we identified several other immune populations including Mϕ, DCs, Treg cells, NK cells and CD4⁺ T cells (FIG. 11).

Mice were also implanted orthotopically (in the mammary fat pad) with the breast cancer cell line PyMT. When tumors were palpable (approx 20 d after implantation) we treated mice with cisplatin+/−loss of IL-33 signaling and followed tumor growth. We found that loss of IL-33 function significantly reduced tumor burden at the end of the experiment 3-fold compared to chemotherapy alone showing that IL-33 function limited chemotherapy efficacy in this model of breast cancer.

### Loss of MSM IL-33 Production Alters the Tumor Immune Microenvironment Driving Inflammatory T Cell Maturation

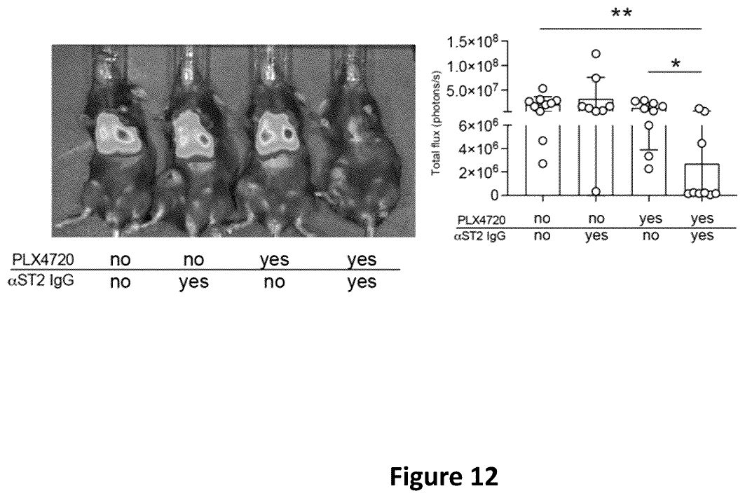

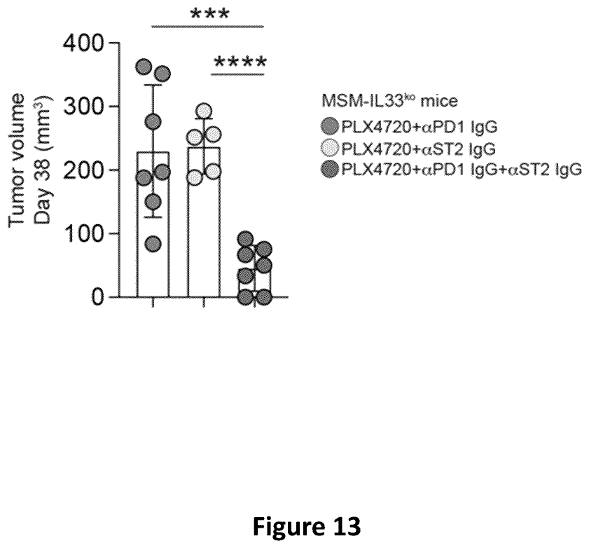

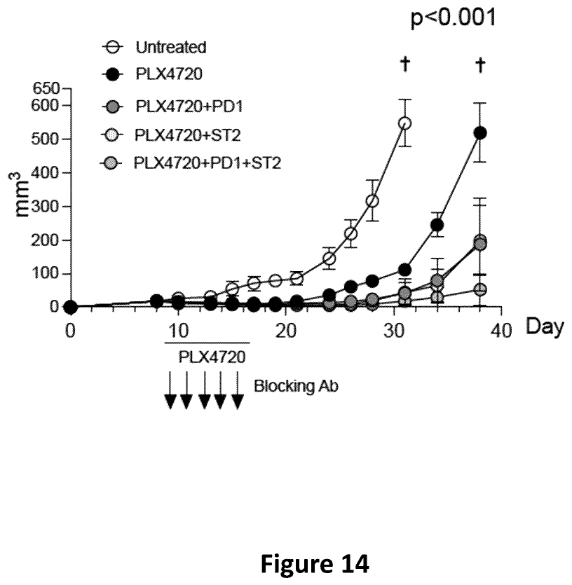

The data show that loss of MSM IL-33 enhanced the inflammatory programs of CD8⁺ T cells in the TME after PLX4720 therapy. To test for the role of CD8⁺ T cells in the observed enhancement of tumor control in MSM-IL-33^{KO} after treatment with the BRAF inhibitor, CD8⁺ T cells were depleted during the course PLX4720 treatment. Deletion of CD8⁺ T cells alone led to a non-significant increase in tumor volume compared to control mice. Importantly, deletion of CD8⁺ T cells during therapy led to a complete loss of the benefit observed in tumor control in MSM-IL-33^{KO} mice after PLX4720 treatment, showing that the improved efficacy of PLX4720 is entirely dependent on improved CD8⁺ T cell function in the TME. We next asked if the enhancement in effector CD8⁺ T cell function would lead to protection from further tumor challenge. To test this, we treated wildtype B6 mice with established tumors with a combination of αST2 IgG i.v.+/−PLX4720 i.p. One day after therapy ended, we surgically excised the tumors and 2 days later we challenged mice with 10⁶ YUMM tumor cells i.v. that were modified to express luciferase (YUMM^{LUC}). Two weeks after YUMM^{LUC} injection we imaged the mice for luciferase activity as a measure of tumor growth. In control mice there was significant tumor burden in the lung, showing that the YUMM^{LUC} cells could actively metastasize and grow under control conditions (FIG. 12). Mice treated with either PLX4720 or αST2 IgG alone showed no reduction in lung tumor burden compared to controls; however, mice receiving PLX4720 and αST2 IgG showed significant resistance to tumor growth (FIG. 12). Thus collectively, the data provide strong evidence that loss of MSM IL-33 production in the TDLN enhances anti-tumor CD8⁺ T cell responses driving potentially durable tumor control and protection from metastatic rechallenge. Since inflamed (i.e. hot) tumors generally exhibit improved responsiveness to checkpoint inhibitor therapy in the clinic, we then asked if combinatorial targeting of BRAF^{V600E}, ST2, and PD-1 would provide superior therapeutic efficacy compared to BRAF^{V600E} inhibition alone. In the untreated control group, all mice were euthanized due to tumor growth on day 30, demonstrating the aggressiveness of the YUMM model (FIG. 14). While PLX4720 treatment alone significantly improved survival, tumor growth rebounded when the mice were removed from therapy and there was 100% mortality on day 38 (FIG. 14). Combination of PLX4720 with either αST2 IgG or αPD-1 IgG had a comparable effect on tumor growth with both groups showing mean tumor volume of approximately 200 mm³ on day 38, a significant improvement in tumor control over PLX4720 alone (FIG. 13 and FIG. 14). However, the combination of all three targeted therapies exhibited the greatest efficacy, with a 4-fold reduction in mean tumor volume compared to the dual treatment arms of the experiment (FIG. 13 and FIG. 14). Thus, the data show that targeting of the immune feed-back vulnerabilities created by IL-33 induction in the TDLN can be exploited potentiate the efficacy of standard of care therapies in subcutaneous melanoma.

IL-33 Responsive T Regs are Required to Limit Tumor Growth after Chemotherapy

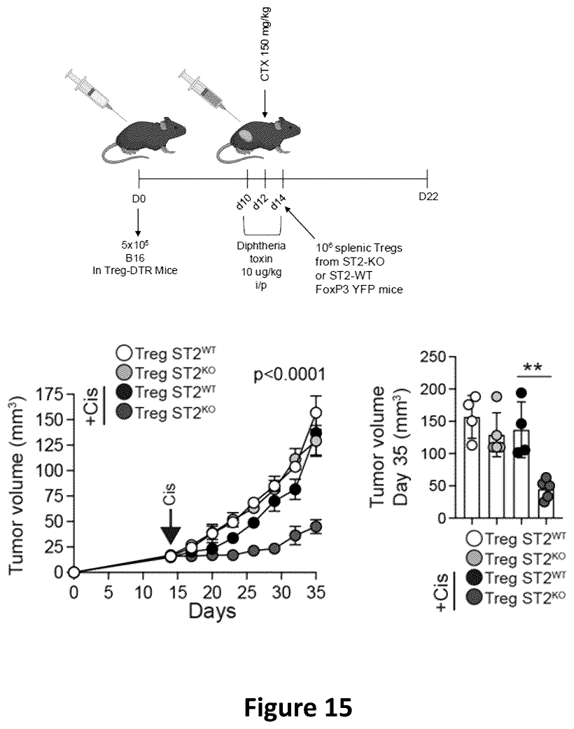

We also performed a depletion and rescue experiment to test if ST2 expression on Treg cells is required for tumor resistance to therapy. Injection of Foxp3^{DTR} mice with diphtheria toxin results in a rapid loss of FoxP3⁺ Treg cells. Thus, we depleted Treg cells in Foxp3^{DTR} mice replacing them with exogenous Treg cells at the height of the TDLN response (i.e. 48 hours post therapy) from either wild type mice or mice deficient in ST2 (B6.Il1rl1^{-/-}) testing the impact of the transferred Treg cells on tumor responses to therapy. For this, we injected Foxp3^{DTR} mice with diphtheria toxin 13 days after sub-cutaneous implantation of 10⁵ B16 cells, followed 6 hours later by injection of cisplatin i.p. Forty-eight hours after cisplatin treatment, (at the time of maximal apoptotic cell trafficking in the TDLN we adoptively transferred 10⁶ Treg cells from either B6.Il1rl1^{-/-} or B6 mice and tested the effect on tumor growth. Long term depletion of Treg cells causes massive autoinflammatory disease; however, while we observed systemic depletion of Treg cells, our short-term depletion protocol did not cause observable pathology or immune activation. Moreover, we found that adoptive transfer of Treg cells did not impact tumor growth in the absence of cisplatin therapy regardless of ST2 status (FIG. 15). If mice received ST2-wildtype Treg cells after cisplatin treatment tumor growth patterns were identical to control groups; however, mice receiving ST2-knock out Treg cells showed significant responses to cisplatin, with a plateau in tumor growth for 7 days after therapy and 3-fold smaller tumors at day 35 (FIG. 15). Thus, cumulatively the data show that MSM IL-33 is required for Treg cell transcriptional maturation and accumulation in TDLN after therapy, and this response is critical for Treg cell activation and tumor resistance to therapy.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Ravishankar, B. et al. Tolerance to apoptotic cells is regulated by indoleamine 2,3-dioxygenase. Proc Natl Acad Sci USA (2012).
2. Ravishankar, B. et al. The amino acid sensor GCN2 inhibits inflammatory responses to apoptotic cells promoting tolerance and suppressing systemic autoimmunity. Proc Natl Acad Sci USA (2015).
3. Ravishankar, B. et al. Marginal zone CD169+ macrophages coordinate apoptotic cell-driven cellular recruitment and tolerance. Proc Natl Acad Sci USA (2014).
4. Shinde, R. et al. Apoptotic cell-induced, TLR9-dependent AhR activity is required for immunological tolerance and suppression of systemic lupus erythematosus in mice and humans. Nature Immunology (2018).
5. Elizabeth E. Gray and Jason G. Cyster. Lymph node macrophages. J Innate Imm. 2012.
6. Firas Hamdan and Vincenzo Cerullo. Cancer immunotherapies: A hope for the incurable? Front. Mol. Med. 2023.
7. Mai, T. T., Nguyen, P. G., Le, M T. et al. Discovery of small molecular inhibitors for interleukin-33/ST2 protein-protein interaction: a virtual screening, molecular dynamics simulations and binding free energy calculations. Mol Divers 26, 2659-2678 (2022). https://doi.org/10.1007/s11030-021-10359-4.
8. Ramadan et al. From proteomics to discovery of first-in-class ST2 inhibitors active in vivo, JCI Insight. 2018; 3(14):e99208.
9. Le M T, Mai T T, Huynh P N H, Tran T D, Thai K M, Nguyen Q T. Structure-based discovery of interleukin-33 inhibitors: a pharmacophore modelling, molecular docking, and molecular dynamics simulation approach. SAR QSAR Environ Res. 2020 December; 31(12):883-904.

1. A method of treating cancer in a subject comprising downregulating IL-33 activity in the subject.

2. The method of claim 1, in combination with a cancer therapy.

3. The method of claim 2, wherein the cancer therapy is chemotherapy.

4. The method of claim 2, wherein the cancer therapy is radiotherapy.

5. The method of claim 2, wherein the cancer therapy is immunotherapy.

6. The method of claim 2, wherein the cancer therapy is PD-1 blockade.

7. The method of claim 2, wherein the cancer therapy is targeted therapy.

8. The method of claim 2, wherein the cancer therapy is any combination of two or more of chemotherapy, radiotherapy, immunotherapy, PD-1 blockade, and targeted therapy.

9. The method of claim 1, wherein downregulating IL-33 activity in the subject comprises administering an inhibitor of IL-33/ST2 interaction to the subject.

10. The method of claim 9, wherein the inhibitor is an antibody against ST2.

11. The method of claim 10, wherein the inhibitor is an antibody against IL-33.

12. The method of claim 10, wherein the inhibitor is a small molecule against ST2 or IL-33.

13. The method of claim 1, wherein downregulating IL-33 comprises genome editing of the subject's cells to decrease transcription or expression of IL-33.

14. The method of claim 1, wherein the cancer is Acute lymphoblastic leukemia (ALL), Acute myeloid leukemia (AML), Adrenal gland, Anal, Bile duct, Bladder, Bone, Brain and spinal cord, Breast, Cervical, Chondrosarcoma, Chordoma, Chronic lymphocytic leukemia (CLL), Chronic myeloid leukemia (CML), Colorectal, Esophageal, Eye, Fallopian tube, Gallbladder, Gestational trophoblastic disease, GISTs, Hodgkin lymphoma, Hypopharyngeal, Kaposi sarcoma, Kidney, Laryngeal, Leukemia, Liver, Lung, Merkel cell carcinoma, Mesothelioma, Metastatic, Multiple myeloma, Myelodysplastic syndromes, Myeloproliferative neoplasms, Nasal and paranasal sinus, Nasopharyngeal, Neuroblastoma, Neuroendocrine tumours, Non-Hodgkin lymphoma, Oral, Oropharyngeal, Osteosarcoma, Ovarian, Pancreatic, Parathyroid, Penile, Pituitary gland tumours, Prostate, Renal pelvis and ureter, Retinoblastoma, Rhabdomyosarcoma, Salivary gland, Skin—melanoma, Skin—non-melanoma, Small intestine, Soft tissue sarcoma, Stomach, Testicular, Thymus, Thyroid, Uterine, Vaginal, Vulvar, or Wilms tumour.

15. A pharmaceutical composition for the treatment of cancer comprising an inhibitor of IL-33/ST2 interaction along with a pharmaceutically acceptable carrier.

16. The pharmaceutical composition of claim 15, further comprising one or more agents for chemotherapy, radiotherapy, immunotherapy, PD-1 blockade, and targeted therapy.

17. A pharmaceutical composition for the treatment of cancer comprising reagents for genomic editing of the subject's cells to decrease transcription or expression of IL-33.
